SEQUENCE LISTING (4 sequences)

SEQ ID NO: 1 - DNA, 783 bp, escherichia coli and Mus sp.
gcccaggtgaaactgcagcagtctggggctgaattggtgaggcctggggcttcagtgaagttgtcctgcaaggcttctggctacagcttcaccgtctactatatttactgggtgaaacagaggcctggacaagcccttgagtggattggagagattaatcctagcaatggtggtactaacttcaatgaaaagttcaagaccaaggccacactgactgtagacaaatccaccagcacagtctacatgcaactcagcagcctgacatctgaagactctgcggtctattactgtacaagatgggggttacgacgagggtttgcttactggggccaagggaccacggtcaccgtctcctcaagtggaggcggttcaggcggaggtggctctggcggtggcggatcggacatcgagctcactcagtctccaaaatccatgtccatgtcagtaggagagagggtcaccttgagttgcaaggccagtgagaatgtgggtactcatgtatcctggtatcaacagagaccagaggagtctcctaaactgctgatatacggggcatccaaccggtacactggggtccccgatcgcttcacaggcagtggctctgcaacagatttcactctgaccatcagcaatgtgcaggctgaagaccttgcagattatcactgtggacagacttacagctatccattcacattcggcttggggacaaagttggaaataaaacgggcggccgcaggtgcgccggtgccgtatccggatccgctggaaccgcgtgccgcatag

SEQ ID NO: 2 - protein, 260 aa, Escherichia coli and Mus sp.
AQVKLQQSGAELVRPGASVKLSCKASGYSFTVYYIYWVKQRPGQALEWIGEINPSNGGTNFNEKFKTKATLTVDKSTSTVYMQLSSLTSEDSAVYYCTRWGLRRGFAYWGQGTTVTVSSSGGGSGGGGSGGGGSDIELTQSPKSMSMSVGERVTLSCKASENVGTHVSWYQQRPEESPKLLIYGASNRYTGVPDRFTGSGSATDFTLTISNVQAEDLADYHCGQTYSYPFTFGLGTKLEIKRAAAGAPVPYPDPLEPRAA

SEQ ID NO: 3 - protein, 16 aa, Escherichia coli and Mus sp.
KASGYSFTVYYIYWVK

SEQ ID NO: 4 - protein, 14 aa, Escherichia coli and Mus sp.
TRWGLRRGFAYWGQ

(19) United States
(12) Patent Application Publication
(10) Pub. No.: US 2006/0229438 A1
(43) Pub. Date: Oct. 12, 2006

(54) Monoclonal antibody derived peptide inhibitors for mycobacterial dna gyrase

(75) Inventors: Valakunja Nagaraja, KARNATAKA (IN); Uijini Havaldar, Karnataka (IN); Bhairab Nath, Maharashtra (IN)

Correspondence Address: LADAS & PARRY, 26 WEST 61ST STREET, NEW YORK, NY, 10023, US

(73) Assignee: LUPIN LTD., MAHARRASHTRA (IN)

(21) Appl. No.: 10/527,834

(22) PCT Filed: Sep. 20, 2002
(86) PCT No.: PCT/IN02/00192

§ 371 Date: Oct. 25, 2005

(51) Int. Cl. A61K 39/40; C12P 21/06; C12N 5/06; C07K 16/12

(52) U.S. Cl. 530/388.4; 424/168.1; 435/69.1; 435/326; 435/320.1

(57) ABSTRACT

The present invention relates to the development of monoclonal antibodies that specifically inhibit DNA gyrase from M. tuberculosis. M. smegmaris and possibly from other related bacterial species. More particularly, it has been shown that the inhibition of the enzyme is by a hitherto unknown and novel mechanism. The present invention also relates to a DNA sequence of single chain antibody consisting of complementarity determining regions of mAb. The monoclonal antibody, single chain antibody and peptides derived thereof could be useful for developing lead molecules for tuberculosis therapy. The antibodies and derived materials could be useful for a variety of purposes, including diagnosis of mycobacterial infections. The present invention also relates to the modification of antibodies and derived materials for use against diverse microbial infections and other potential applications derived thereof.

The present invention provides a monoclonal antibody (mAb), an engineered single chain antibody (scFv), peptides carrying complementarity determining regions (CDR) and peptides derived from CDRs and framework regions, all of which specifically inhibit mycobacterial DNA gyrase activity.

### BACKGROUND OF THE INVENTION

Mycobacterium tuberculosis, the causative agent of tuberculosis is responsible for millions of deaths worldwide each year. One third of the global population is infected with tuberculosis with 6 million new cases reported every year. 20% of adult deaths and 6% of infant deaths are attributable to tuberculosis (C. Dye et al., J. Am. Med. Ass., 1999, 282 677-686). The synergy between tuberculosis and the AIDS epidemic, and the emergence of multi-drug-resistant strains of M. tuberculosis has resulted in the urgent need for new drugs to combat M. tuberculosis infections (S. H. E. Kaufmann et al., Trends Microbiol., 1993, 1, 2-5; B. R. Bloom et. al., N. Engl. J. Med., 1998, 338 677-678).

DNA gyrase is an essential topoisomerase, found exclusively in bacteria The enzyme belongs to the family of type II DNA topoisomerases, a group of enzymes that catalyze interconversions of different DNA topological forms. The enzyme is a chosen molecular target for the development of new antibacterials due to its ability to supercoil DNA, a property not shared by other topoisomerases (A. Maxwell, Trends Microbiol., 1997, 5, 102-109; J. J. Champoux, Annu. Rev. Biochem., 2001, 70, 369-413 ; A. D. Bates et al., In DNA topology, Oxford University Press, Oxford, 1993; R. J. Reece et al., CRC Cit. Rev. Biochem. Mol. Biol., 1991, 26,335-375).

The DNA gyrase has been extensively studied from E. coli. The enzyme from E. coli is composed of two subunits, DNA gyrase A protein (GyrA) and DNA gyrase B protein (GyrB), the active form being an A₂B₂ heterotetrainer. GyrA comprises two domains; an N-terminal domain (64 kDa) that contains the active-site tyrosine residue involved in DNA cleavage, and C-terminal domain that is involved in the wrapping of a segment of DNA around A₂B₂ complex. The GyrB protein also consists of two domains: a 43 kDa N-terminal domain, containing the site of ATP binding and hydrolysis, whereas 47 kDa C-terminal domain is involved in DNA binding and interaction with GyrA subunit (R. J. Reece et al., CRC Crit. Rev. Biochem. Mol. Biol., 1991, 26, 335-375).

A large number of E. coli DNA gyrase inhibitors, which belong to diverse classes, have been characterized. Amongst them, quinolones and coumarins were the first ones to be characterized and most extensively studied for their inhibitory activity.

The quinolones are synthetic class of compounds, which interfere with the processes of rejoining the double strand breaks in DNA (K. Drlica et. al., Biochemistry, 1988, 27, 2253-2259). New generation of quinolones especially fluoroquinolones have found wide applications clinically for variety of bacterial infections (D. C. Hooper, Emerg Infect Dis., 2001, 7, 337-41). Although mycobacteria have been found to be naturally less susceptible to quinolones than other bacteria (J. S. Wolfson et. al., Clin. Microbiol. Rev., 1989, 2, 378-424), new quinolones have been demonstrated to be active against mycobacterial infections (J. M. Woodcock et. al., Antimicrobial. Agents Chemother., 1997, 41, 101-116). Amongst them moxifloxacin and sparfloxacin have been found to be most active against mycobacteria (S. H. Gillespie et. al., J. Antimicrob. Chemother., 1999, 44, 393-395; U. H. Manjunatha et. al., Nucleic Acids Res., 2002, 30, 2144-2153).

The coumarins are naturally occurring antibiotics, which affect ATPase activity of gyrase (H. Hoeksema et. al., J. Am. Chem. Soc., 1985, 78, 6710-6711; R. J. Lewis et. al., EMBO J., 1996, 15, 1412-1420). Cyclothialidines, a class of cyclic peptides have been characterized to inhibit DNA gyrase activity in a fashion analogous to that of coumarins (N. Nakada et. al., Antimicrob. Agents Chemother., 1994, 38, 1966-1973). In addition, two proteinaceous poisons, microcin B17 (F. Baquero et. al., J. Bacteriol., 1978, 135, 342-347) and CcdB (P. Bernard et. al., J. Mol. Biol., 1992, 226, 735-745) inhibit E. coli DNA gyrase. Most of these characterized inhibitors fall into two groups based on their site of action and mechanism of inhibition, one that affects cleavage-rejoining step and others that inhibit ATP hydrolysis. Supercoiling reaction involves a series of complicated steps, yet the inhibitors described target only two of these steps.

The present invention is based on the premise that the enzyme provides additional opportunities to develop inhibitors, which could affect other steps in the reaction cycle. The potential inhibition of other steps of gyrase reaction has not been explored so far.

Considering the global menace of mycobacterial infections the approach adapted by our laboratory has been to study mycobacterial gyrase and compare its properties with the well-characterized DNA gyrase for development of a mycobacterial enzyme as molecular target for new drug discovery.

For this purpose GyrA and GyrB subunits from M. smegmatis (K. Madhusudan et. al., Microbiology, 1995, 140, 3029-3037) and M. tuberculosis (K. Madhusudan et. al., Biochem. Mol. Biol. Int., 1994, 33 651-660; U. H. Manjunatha et. al., Curr. Sci., 2000, 78, 968-974) have been cloned and over-expressed. The studies on mycobacterial DNA gyrase and comparison of its properties with E. coli enzyme has revealed many differences, leading to the classification of DNA gyrases into two subclasses (U. H. Manjunatha et. al., Curr. Sci., 2000, 78, 968-974 ; M. Chatterji, et. al., J. Biol. Chem., 2000, 275 22888-22894; U. H. Manjunatha et. al., Eur. J. Biochem., 2001, 268, 2038-2046). Unlike E. coli enzyme, mycobacterial gyrase is refractory to the plasmid borne proteinaceous inhibitors CcdB and microcin B17 and exhibits reduced susceptibility to fluoroquinolones (M. Chatterji et. al., J. Antimicrob. Chemother., 2001, 48 479-485; U. H. Manjunatha et. al. Nucleic Acids Res., 2002, 30 2144-215).

Monoclonal antibodies owing to their specificity of interaction have been exploited in biology for variety of purposes leading to various applications. They are routinely used in diagnostics, structure-function analysis and as reagents to study protein-protein interactions. A more recent application is the identification of peptide inhibitors for potential antigens by paratope derived peptide approach. R. B. Tournaire et. al., (EMBO J., 2000, 19, 1525-1533) have identified a peptide that blocks vascular endothelial growth factor (VEGF) mediated angiogenesis, using an anti-VEGF mAb. However, development of neutralizing monoclonal antibodies and design of peptide inhibitors against mycobacterial DNA gyrase is an approach not been carried out for any other topoisomerases.

### SUMMARY OF THE INVENTION

In one aspect, the present invention provides an engineered single chain antibody, viz. a recombinant ScFV:GyraA protein, which inhibits DNA gyrase from M. smegmatis and M. tuberculosis.

In another aspect, the present invention provides a plasmid encoding the recombinant ScFV:GyraA protein, viz. pVNUHMScFv, which inhibits DNA gyrase from M. smegmatis and M. tuberculosis.

In yet another aspect, the present invention provides a DNA sequence of the plasmid encoding the recombinant ScFV: GyraA protein as shown in Seq. ID #1, which inhibits DNA gyrase from M. smegmatis and M. tuberculosis.

In yet another further aspect, the present invention provides an amino acid sequence of the recombinant ScFV:GyraA protein as shown in Seq. ID #2, which inhibits DNA gyrase from M. smegmatis and M. tuberculosis.

In yet another embodiment of the invention, said engineered single chain antibody contains an amino acid sequences which inhibit the activity of DNA gyrase from M. smegmatis and M. tuberculosis, said amino acid sequences having the Seq. ID #3 and Seq. ID #4 respectively.

In a further aspect, the present invention provides monoclonal antibodies, viz. MSGyrA:C3 and MSGyrA:H11, which inhibits DNA gyrase from fluoroquinolone resistant M. smegmatis and M. tuberculosis.

In yet another further aspect, the present invention provides hybridoma cell lines C3B3 and H11E1, which secrete the monoclonal antibodies, MSGyrA:C3 and MSGyrA:H11, which also inhibits DNA gyrase from M. smegmatis and M. tuberculosis.

The monoclonal antibody (mAb) described in this invention has been generated against GyrA subunit of M. smegmatis DNA gyrase. The mAb cross reacts with GyrA subunit from fast and slow growing mycobacteria (U. H. Manjunatha et. al., Eur. J. Biochem., 2001, 268, 2038-2046). The invention describes the inhibition of DNA supercoiling activity catalyzed by M. tuberculosis DNA gyrases by full-length mAb and its Fab and single chain antibody (scFv) fragments. The present invention also describes inhibition of DNA gyrase activity by peptides derived from scFv. The invention also deals with novel mechanism of DNA gyrase inhibition is distinct from that of other known DNA gyrase inhibitors.

### DESCRIPTION OF THE FIGURES

FIG. 1A: Specificity of interaction of mAb.

FIG. 1B: Effect of mAbs on mycobacterial DNA gyrase supercoiling activity.

FIG. 2A: Effect of MsGyrA:C3 on DNA binding.

FIG. 2B: Effect of MsGyrA:C3 on DNA cleavage.

FIG. 2C: Effect of MsGyrA:C3 on ATP hydrolysis.

FIG. 2D: Effect of MsGyrA:C3 on ATP independent DNA relaxation reaction of mycobacterial DNA gyrase.

FIGS. 3A and 3B: Effect of MsGyrA:C3 on quinolone resistant M. smegmatis DNA gyrase.

FIG. 3C Effect of MsGyrA:C3 on quinolone resistant M. tuberculosis DNA gyrase.

FIG. 3D Effect of MsGyrA:H11 on quinolone resistant M. smegmatis DNA gyrase.

FIG. 4A: Surface plasmon resonance spectroscopy showing affinity of interaction of scFv:GyrA with GyrA as compared to that of IgG or Fab fragments.

FIG. 4B: Comparison of the inhibition of M. smegmatis DNA supercoiling activity of scFv:GyrA polypeptide and IgG or Pab fragments.

FIG. 4C: Effect of scFv:GyrA on M. tuberculosis DNA gyrase.

FIG. 5: Nucleotide and predicted amino acid sequences of scFv:GyrA gene;

FIG. 6 : Peptide sequences of the engineered single chain antibody, scFv:GyrA and the effect of paratrope derived peptides, CDR:H1 and CDR:H3 on M. smegmatis DNA gyrase supercoiling activity.

FIG. 7: Effect of scFv:GyrA and CDR:peptides on ciprofloxacin resistant M. smegmatis DNA gyrase supercoiling activity.

### DETAILED DESCRIPTION OF THE INVENTION

The DNA supercoiling activity is essential for bacterial survival. The invention pertains to development of monoclonal antibodies (mAb) and its single chain antibody (ScFv) to neutralize mycobacterial DNA gyrase supercoiling activity. The mAb henceforth is termed as MsGyrA:C3 and its single chain antibody fragment is referred as scFv:GyrA The other mAb described in the present invention is MsGyrA:H11. The monoclonal antibodies can be obtained from culture supernatant of the secreting hybridoma cell line in the tissue culture medium or from the ascitic fluid by injecting hybridoma cells into the peritoneal cavity of mouse. The mAb can be purified by protein-A or protein-G sepharose affinity column or by any other chromatographic techniques. The antigen binding fragments of mAb cain be obtained by papain or pepsin digestion of IgG or other methods.

The present invention provides opportunities for utilization of DNA gyrase as drug target by employing novel strategies. The polypeptide inhibitor (mAbs) characterized here selectively inhibits DNA gyrase from M. smegmatis and M. tuberculosis. Since mAb cross-reacts with GyrA from other species of mycobacteria (M. bovis, M. leprae and M. avium; U. H. Manjunatha et. al., Eur. J Biochem., 2001, 268, 2038-2046), it opens up the avenue for the design of specific lead molecules targeted against other mycobacterial infections. The present investigation brings to the fore the distinct mechanism of DNA gyrase inhibition by mAb from that of quinolone or coumarin class of drugs. Absence of cross-resistance to fluoroquinolone resistant DNA gyrase by mAb, warrants the pursuit of this strategy further as it could aid in countering the drug resistance problem. In addition, these novel inhibitors could an invaluable tool in elucidating the various steps of supercoiling reaction, which in turn would facilitate rational design of lead molecules.

The invention described includes scFv:GyrA gene sequence encompassing complementarity determining regions (CDRs) as well as framework regions of both heavy and light chains regions. The scFv gene sequence can be obtained by sequencing cDNA from the mAb secreting hydridoma cell line. The scFv polypeptide in the invention was cloned in phagemid vector and expressed on the surface of phages as well as soluble protein. The scFv can also be cloned in other bacterial expression or any other eukaryotic expression system. In the present investigation scFv was cloned with E-tag and the tag specific antibody is used as an affinity handle. Similarly, scFv can be further cloned with or without other polypeptide tags that can be used for purification and detection of the scFv. The scFv illustrated in the present invention can be modified by site directed or random mutagenesis to increase the affinity of interaction or to alter the specificity of interaction by standard molecular biological techniques. The scFv can be expressed in Mycobacterium sp. using suitable expression system to understand the in vivo role of DNA gyrase and to analyze in vivo toxicity of mAb.

The binding strategy utilized by mAbs can be analyzed to develop synthetic peptides with similar binding properties. This has been accomplished in a number of systems where synthetic peptides have been derived directly from the amino acid sequences of CDRs and demonstrated to have binding properties similar to those of the intact antibody. This strategy provides analysis of intermolecular interactions involved in binding and can lead to development of novel binding moieties with predictable activities. A further embodiment of this invention includes design and synthesis of a mimetic from the CDR of an antibody having gyrase inhibitory activity. The peptides could be covalently modified for stability and efficacy. The present invention also includes futuristic design and synthesis of non-peptido mimetics against DNA gyrase derived from paratope derived peptides. Further, the invention can also be used to develop inhibitors with broad-spectrum applications. In a further embodiment of this invention, this approach of developing peptide inhibitors can be used for other topoisomerases as a therapeutic strategy.

The mAb or fragments of antibody described in the invention can also be used as an affinity handle for purification of mycobacterial DNA gyrase. The specificity of interaction of monoclonal antibody and its derivatives to mycobacterial GyrA subunit could also be explored as potential diagnostic reagents for mycobacterial infections. The mAb fragment can be humanized by replacing murine constant regions with human constant regions. The mAbs described in the invention and their fragments can be used to study gyrase interacting proteins, which could be potential drug targets.

The invention is further described in detail as under:

A. Antibody Production and Characterization

The following experiments were performed to generate M. smegmatis GyrA specific mAbs and to study the specificity of interaction and cross-reactivity pattern.

A1: Generation of Anti-GyrA Monoclonal Antibodies:

To raise monoclonal antibodies (G. Kohler et al., Nature, 1975, 256, 495-497) against M. smegmatis GyrA, 8 weeks old Balb/c mice were immunized with 100 μg GyrA in Freund's complete adjuvant in multiple subcutaneous sites. Mice were boosted at three weekly intervals in incomplete adjuvant for a period of three months, and the immune response was monitored by ELISA using M. smegmatis GyrA coated microtiter (Nunc) plates. The spleenocytes from an immunized mouse were fused with Sp2/o myeloma cell line (1:5 ratio) by the PEG-mediated cell fusion technique. The fused cells were selected in HAT medium The production of antibodies by the clones was assayed by ELISA with the culture supernatant. The clones with consistent and significant reactivity were subcloned to monoclonality by limiting dilution. Two hybridoma clones C3B3 and H11E1 which secrete MsGyrA:C3 and MsGyrA:H11 respectively were further characterized.

A2 : Purification of mAbs:

Hybridoma cells (1×10⁶) were washed with serum free medium two times and injected in mice intraperitonealy without priming the animals. The ascitic fluid was collected from animals after 15-18 days of injection. Ascitic fluid was diluted in start buffer containing 3 M NaCl and 1.5 M Glycine-NaOH pH 8.9, and passed through a protein A Sepharose column previously equilibrated with the same buffer (E. Harlow et. al., “Antibodies: A Laboratory Manual.” Cold Spring Harbor, N.Y., 1988). The elution was done with 100 mM Citrate buffer (elution buffer) (pH 6.0), where IgG₁ elutes at pH 6.0-7.0. The eluate was neutralied immediately with 1.0 M Tris-HCl pH 9.0 (neutralizing buffer). The affinity column was regenerated using 100 mM citrate buffer pH 3.0.

A3: Immunoblotting Techniques:

SDS-PAGE was performed using 8% polyacrylamide separating gels (U. K. Laemmli, Nature, 1970, 227, 680-685). Proteins from the gel were transferred to PVDF membrane in Western transfer buffer (Tris-Cl 25 mM (pH 8.0), Glycine 192 mM and Methanol 20%) for 2 hours at 200 mA using Bio-Rad protein transfer apparatus. The membrane was blocked with 0.4% BSA in PBS, followed by incubation of primary antiserum in PBST-BSA (0.4%) for 2 hours. Bound antibody was detected using an anti-rabbit or anti-mouse horse radish peroxidase conjugate and detected by enhanced chemiluminiscence (Amersham ECL Plus) according to the manufacturer's instructions.

A4 : Experiments to Show That mAb is Specific to Mycobacteria:

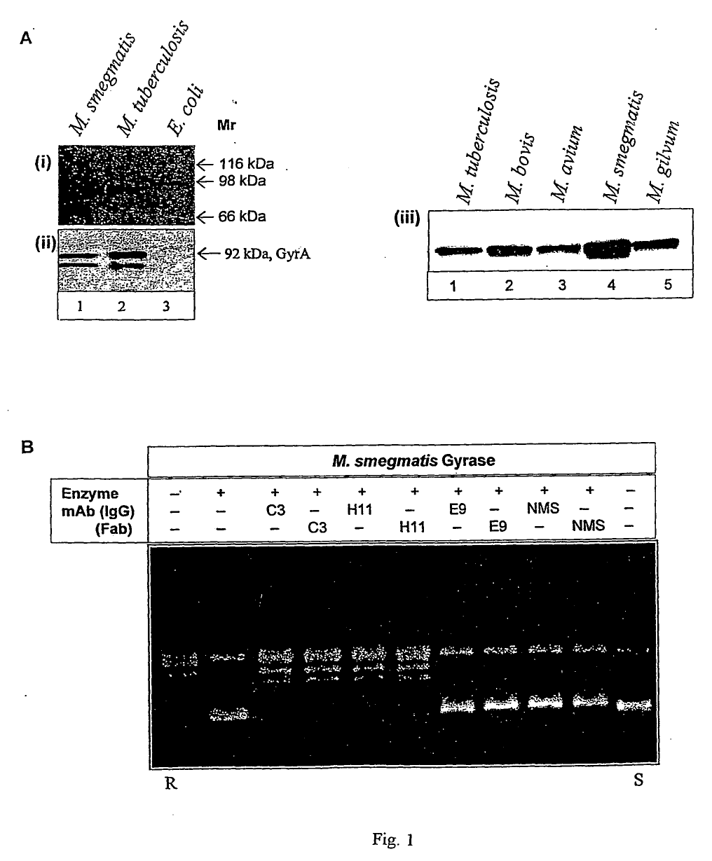

Western Blot analysis using MsGyrA-C3 was performed with purified M. smegmatis, M. tuberculosis and E. coli GyrA proteins. The antibody recognized mycobacterial GyrA subunit but not from E. coil (FIG. 1A). M. tuberculosis and M. smegmatis GyrA show proteolytic degradation to a protein fragment of 78 kDa, which was also recognized by the monoclonal antibody. Similar cross-reactivity pattern is observed with another monoclonal antibody MsGyrA:H11.

Sequence alignment of M. smegmatis GyrA; X84077 (K. Madhusudan et. al., Microbiology, 1995, 140, 3029-3037) with other known mycobacterial DNA GyrA subunits (M. tuberculosis; L27512, H. E. Takiff et. al., Antimicrob. Agents Chemother., 1994, 38, 773-780) and M. leprae; Q57532, H. Fsihi et. al., Proc. Natl. Acad. Sci. USA, 1996, 93, 3410-3415) indicates approximately 90% sequence identity at the amino acid level (U. H. Manjunatha et. al., Curr. Sci., 2000, 78, 968-9). To study the cross-reactivity of the mAbs with gyrases from other mycobacterial species, crude cell lysates were prepared and analyzed by Western Blotting. MsGyrA:C3 recognized GyrA from both fast growing and slow growing mycobacterial species (FIG. 1A). The GyrA subunits of all the species tested have very similar molecular weight. As can be seen from FIG. 1A, (I) shows SDS-PAGE and (ii) shows Western blot analysis of purified GyrA proteins (1 μg/lane). The protein samples were subjected to SDS-PAGE and Western blot was probed with 10 ng/ml of MsGyrA:C3. FIG. 1A (iii) shows Western blot analysis of various slow and fast growing mycobacterial cell free extarcts (20 μg/lane) with MsGyrA:C3. The size of the GyrA sub-unit is indicated and Mr represents protein size markers with indicated molecular mass.

B. Inhibition of Mycobacterial DNA Gyrase Supercoiling Activity

The experiments presented in this section describe the effect of MsGyrA:C3 and MsGyrA:H11 IgG and their Fab fragments on DNA gyrase supercoiling activity.

B1: Preparation of Fab Fragments:

Fab fragments of the IgG were prepared by digestion of the purified IgG with papain. Briefly, 500 μg of purified IgG was incubated in 100 mM sodium acetate buffer, pH 5.5 containing 10 mM β-mercaptoethanol, 1 mM EDTA and 5 μg of papain. Digestion was continued for 12 hours at 37° C. in vacuum and quenched by the addition of 150 mM iodoacetamide. The mixture was dialyzed against PBS, purified by protein-A column and analyzed for digestion by 15% SDS-PAGE.

B2: DNA Supercoiling Assays:

Relaxed DNA was prepared by treating supercoiled pUC18 DNA with E. coli topoisomerase I (R. M. Lyn et. al., Proteins, 1989, 6, 231-239). The supercoiling reaction was carried out in a potassium glutamate buffer (KGB) with 2 mM MgCl₂, 1.8 mM spermidine-HCl, 9 μg/ml yeast t-RNA, 50 μg/ml BSA and 1.4 mM ATP in a 20 μl reaction volume with 300 ng of relaxed DNA. After one hour incubation at 37° C., the reaction was stopped by adding 4 μl of stop buffer (0.6% SDS, 0.2% Bromophenol blue, 0.2% Xylene cyanol FF) and heat inactivated at 75° C. for 15 minutes. The samples were electrophoresed in a 1.2% agarose gel for 10-12 hours at 25 V. The gel was stained with ethidium bromide (0.5 μg/ml). The supercoiling assays with E. coli gyrase were carried out as described by K. Mizuuchi et. al., J. Biol. Chem., 1984, 259, 9199-9201. One unit of gyrase was defined as concentration of enzyme that catalyses the conversion of 300 ng of relaxed pUC18 DNA into completely supercoiled form in 1 hour at 37° C.

B3: Experiments to Show that mAbs Inhibit DNA Gyrase Supercoiling Activity:

M. smegmatis gyrase was preincubated with individual mAbs to allow the formation of the antigen-antibody complex. The mixture was then added to relaxed pUC18 DNA, in supercoiling reaction buffer. The results (FIG. 1B) showed that both MsGyrA:C3 and MsGyrA:H11 inhibited the mycobacterial DNA gyrase supercoiling activity while another mAb MsGyrA:E9 showed no reduction in supercoiling activity. MsGyrA:E9 is also a M. smegmatis GyrA specific mAb, interacts with GyrA in a denatured form (U. H. Manjunatha et. al., Eur. J. Biochem., 2001, 268, 2038-2046). None of these mAbs affected the supercoiling activity of E. coli DNA gyrase. This is in agreement with Western Blotting results that had indicated the absence of cross reactivity with the E. coli enzyme.

As whole IgG was used in these studies, it was possible that inhibition of gyrase activity resulted from steric effects caused by the Fc regions of these mAbs or by cross-linking of adjacent gyrase molecules. To address this possibility, Fab fragments of mAbs were prepared and tested to determine whether these monovalent fragments were also capable of inhibiting DNA gyrase supercoiling activity. As shown in FIG. 1B, Fab fragments of MsGyrA:C3 and MsGyrA:H11 also inhibited the supercoiling reaction. MsGyrA:C3 completely inhibited supercoiling activity at a concentration of 10 nM of IgG and 50 nM of Fab, whereas MsGyrA:H11 whose affinity is slightly lower than MsGyrA:C3 inhibited supercoiling activity completely at a concentration of 50 nM of IgG and 100 nM of Fab.

C. Elucidation of Mechanism of DNA Gyrase Inhibition by MsGyrA:C3

DNA gyrase catalyses inter-conversion of DNA topological forms involving a series of conformational changes in the enzyme. The processes involves the wrapping of DNA around the enzyme, cleavage of DNA in both strands (making a covalent complex of gyrase-DNA), and passage of the segment of DNA through this double strand break. This is coupled to ATP hydrolysis and results in the introduction of negative supercoils (J. J. Champoux, Annu. Rev. Biochem., 2001, 70, 369-413; J. C. Wang, Annu. Rev. Biochem., 1996, 65, 635-692 and references cited therein). The molecular gates open-close alternatively to ensure that DNA entry-breakage-reunion-exit cycle is completed. This section elucidates the mechanism of DNA gyrase inhibition by the monoclonal antibody. The experiments presented for understanding the mode of mAb inhibition are not routine. MsGyrA:C3 inhibited DNA gyrase activity by a mechanism distinct from that of quinolone and coumarin class of inhibitors. Further, it is demonstrated that the enzyme is inhibited at a novel step of the reaction cycle.

C1: Purification of DNA Gyrase:

M. smegmatis and M. tuberculosis DNA gyrase holoenzymes were purified by novobiocin-Sepharose column as described by U. H. Manjunatha et. al., FEMS Microbiol. Letters, 2001, 194, 87-92. Individual mycobacterial gyrase subunits used for some experiments were purified by immunoaffinity column chromatography (U. H. Manjunatha et. al., Nucleic Acids Res., 2002, 30, 2144-2153).

C2: Electrophoretic Mobility Shift Assay:

Electrophoretic mobility shift assays were carried out using a radiolabeled 240 bp DNA fragment encompassing the strong gyrase site from pBR322 (L. M. Fisher et. al., Proc. Natl. Acad. Sci. USA, 1981, 78, 4165-4169). Labeled DNA (0.1×10⁻⁹ M) was incubated with 5 nM mycobacterial gyrase in supercoiling buffer for 30 minutes at 4 ° C. For supershift assays, 5 nM of antibody was added either to enzyme or DNA-enzyme complex. The samples were electrophoresed on a 3.5% native polyacrylamide gel at 4° C. in 0.5× TBE buffer containing 10 mM MgCl₂.

C3: ATPase Assay:

ATPase assays were performed as described previously by M. Chatterji et. al., J. Biol. Chem., 2000, 275, 22888-22894 with 10 Us of purified M. smegmatis DNA gyrase with or without preincubation of 120 μg/ml mAb or normal mouse IgG or 20 μg/ml novobiocin at 4° C. for 30 minutes.

C4: Cleavage and Religation Assay:

The cleavage reaction (12.5 μl) was carried out in supercoiling buffer with linear radiolabeled 240 bp DNA fragment with varying concentrations of DNA gyrase in presence of 30 μg ml⁻¹ ciprofloxacin as described by U. H. Manjunatha et. al., Nucleic Acids Res., 2002, 30, 2144-2153. The samples were analyzed on 6% denaturing polyacrylamide gel electrophoresis.

C5: DNA Relaxation Assay:

Relaxation assays were carried out with 10 U of enzyme in the supercoiling buffer devoid of ATP using supercoiled DNA as substrate and incubated for 6 hours at 37° C.

C6: Experiments to Show That the Mechanism of DNA Gyrase Inhibition by MsGyrA:C3 is Distinct from Quinolone and Coumarin Class of Inhibitors

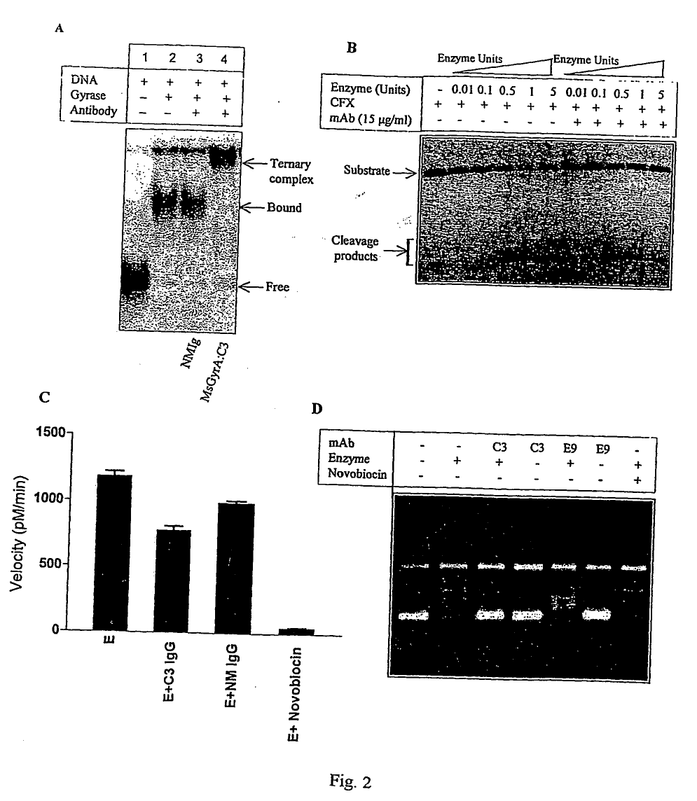

The experiments presented in this section of the invention address the mechanism of inhibition of DNA gyrase activity by mAb. We demonstrate that the mAb binding did not affect gyrase subunit-subunit interaction or holoenzyme-DNA interaction. Instead, a ternary complex of gyrase-DNA-mAb is formed (FIG. 2A). The ternary complex is competent to perform quinolone induced DNA cleavage (FIG. 2B). Further, religation of the cleaved G-segment is also not affected. The conformational changes induced in the quinolone stabilized cleavable complex (S. C. Kampranis et. al., J. Biol. Chem., 1998, 273, 22606-22614) did not affect mAb binding. In the presence of mAb, trapping of cleaved DNA complex was not observed, implying that the mechanism of inhibition by mAb is distinct from that of quinolone class of drugs.

Binding of mAb to mycobacterial gyrase did not abolish DNA stimulated ATP hydrolysis indicating that mAb mediated inhibition employs distinct mechanism than coumarin class of inhibitors (FIG. 2C). Binding of MAb:C3 IgG to gyrase abolishes the ATP independent relaxation of negatively supercoiled DNA (FIG. 2D). Similar results were observed with Fab fragments of the mAb as well. Unlike coumarins that inhibit only the supercoiling reaction and not DNA relaxation activity, mAb inhibited both DNA supercoiling as well as DNA relaxation activities, re-emphasizing the distinct mode of mAb action.

D. Inhibition of Quinolone Resistant M. smegmatis and M. tuberculosis DNA Gyrase by MsGyrA:C3 and MsGyrA:H11

Since the inhibition by mAb is by a mechanism very different from known modes of inhibition, DNA gyrase from quinolone resistant strain should be susceptible to antibody mediated inhibition. In this section, the above hypothesis is tested using quinolone resistant M. smegmatis and M. tuberculosis DNA gyrases.

D1: Experiments to Show that mAb is Effective Against Quinolone Resistant Mycobacterial DNA Gyrase

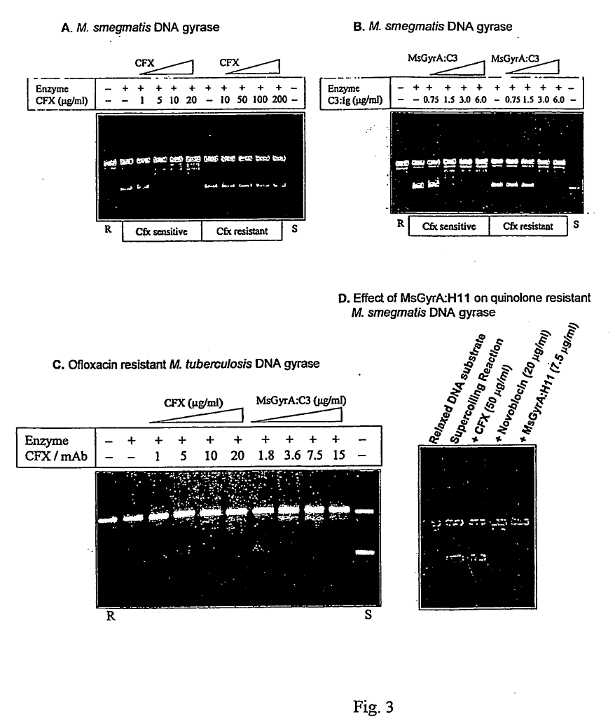

DNA gyrase from ciprofloxacin resistant (MIC₅₀ 64 μg/ml) M. smegmatis mc²155 strain was used for the experiments. As expected, the enzyme from quinolone sensitive strain showed an IC₅₀ of ˜5 μg/ml of ciprofloxacin, whereas resistant strain showed no inhibition even at 400 μg/ml (FIG. 3A). DNA supercoiling activity was inhibited at 3 μg/ml and 6 μg/ml concentrations of MsGyrA:C3 for quinolone sensitive (D^{S}) and quinolone resistant (D^{R}) enzymes respectively (FIG. 3B). The twofold difference in the mAb concentration between D^{S} and D^{R} enzymes is attributed to reduced specific activity of D^{R} enzyme. DNA gyrase from ofloxacin resistant, highly virulent clinical isolate of M. tuberculosis (ICC-222) was also assayed for the effect of mAb. The purified enzyme has an IC₅₀ of ˜10 μg/ml for ciprofloxacin, where as the MsGyrA:C3 inhibited DNA gyrase supercoiling activity at 3.0 μg/ml, similar to that of M. smegmatis enzyme (FIG. 3C). The absence of cross-resistance essentially emphasizes the mode of action of mAb to be distinct to that of quinolones. Similar to MsGyrA:C3, MsGyrA:H11 also inhibited ciprofloxacin resistant M smegmatis DNA gyrase (FIG. 3D). These data confirm the novel inhibition mechanism of gyrase by mAb. Absence of cross-resistance to fluoroquinolone resistant DNA gyrase by mAb, warrants the study of MsGyrA:C3 further as it could aid in countering the drug resistance problem.

E. Cloning, Sequencing and Expression of a DNA Sequence Encoding for Neutralizing Antibody Gene and Design of Bioactive Peptides

This example describes the cloning and expression of a nucleic acid sequence coding for a DNA gyrase neutralizing single chain antibody, scFv:GyrA. Based on the inhibition of gyrase by scFv:GyrA and utilizing sequence of the antibody, bioactive peptides were designed and their inhibition of mycobacterial DNA gyrase was tested.

E1: Cell Culture and Isolation of RNA:

Total RNA was isolated from the actively secreting mAb:C3 hybridoma cell line. Briefly, confluent hybridoma cells (3×10⁸) were washed with ice cold IMDM medium and total RNA was extracted using TRIzol reagent (Life technologies Inc). RNA was purified using RNeasy QUIAGEN as per the manufacturer's protocol, The quality of RNA was confirmed by electrophoresis in a 1% formaldehyde agarose gel.

E2 : First-Strand cDNA Synthesis:

The first-strand cDNA was synthesized from total RNA using the reverse transcription reaction (RT). For annealing, 5 μg of total RNA was incubated with 0.2 μg/ml of random hexamer oligonucleotide in a 10 μl reaction volume at 70° C. for 5 minutes, followed by immediate chilling on ice. The annealed mix was incubated with 1 mM dNTP and 20 Units of Moloney Murine Leukemia Virus reverse transcriptase, (M-MuLV RT obtained from MBI Fermentas) in RT reaction buffer. The reaction was carried out at 25° C. for 10 minutes followed by 37° C. for one hour and then stopped by heat denaturation at 70° C. for 10 minutes.

E3: Construction of the scFv Gene Fragment:

The single chain Fv of MsGyrA:C3 was cloned using the Recombinant Phage Antibody System (RPAS, Amersham Pharmacia Biotech) as per manufacturer's instructions. The VH and VL antibody genes were amplified in two separate reactions using specific mixture of heavy chain primers and light chain (κ) specific primers with Taq DNA polymerase. The heavy chain amplification reaction yielded a fragment approximately 340 base pairs in length, while the corresponding light chain fragment was 325 base pairs. The gel purified heavy and light chain fragments were assembled into a single gene using a DNA linker fragment. The assembly reaction ultimately produced a small amount of the single-chain Fv gene where the VH region was linked to the VL region via a sequence encoding a (Gly₄Ser)₃ linker. The scFv DNA fragment was ˜750 base pairs in length, The assembled antibody scFv DNA fragment was amplified by PCR with a set of oligonucleotide primers that introduced Sfi I and Not I sites for cloning into the pCANTAB 5 E vector. The scFv gene product was subsequently cloned in the Not I and Sfi I sites of the pCANTAB 5E phagemid vector (Amersham Pharmacia Biotech) to obtain pVNUHMscFv.

E4: Expression and Selection of Phage scFv:

A recombinant phage antibody library for MsGyrA:C3 was generated by transformation of E. coli TG-1 cells with pVNUHMscFV as described above. Infection by M13-K07 (4×10¹⁰ pfu for 10 ml of early log culture) helper phage allowed packaging of the recombinant phagemid into phage-expressing antibody. The antigen-reactive phages were enriched through solid phase panning with GyrA coated plate. Briefly, microtiter wells (Nunc) coated with M. smegmatis GyrA (200 ng/well) were incubated with phages for 2 hours at 37° C. The unbound phages were washed thrice with 10 mM phosphate buffer, followed by infection with TG1 cells. Infected TG-1 cells were plated on selection medium Phages rescued from the individual plaques after biopanning were subsequently screened by ELISA using M. smegmatis GyrA bound microtitre plates with culture supernatants. Positive wells were detected with horseradish peroxidase conjugated anti-M13 antibody (Amersham pharmacia). The antigen positive clones were rescued by helper phage infection and screened for binding by ELISA against M. smegmatis GyrA. The phages capable of binding M. smegmatis GyrA were used to infect E. coli HB2151 for generation of soluble scFv.

E5: Expression and Detection of Soluble scFv:

HB2151 cells were infected with the recombinant antigen positive phages for soluble scfv production. Infected E. coli HB2151 cells grown to an A₅₀₀ of 0.6 as per manufacturer's instructions and induced with 1 mM IPTG and grown at 25° C. for 11 hours. The induced culture was pelleted down and the culture supernatant was filter sterilized. The culture supernatant fractions were screened for the presence of scFv by ELISA against M. smegmatis GyrA bound microtitre plates and further detected with horseradish peroxidase conjugated anti-E tag antibody (Amersham Pharmacia Biotech).

E6 : Purification of scFv Fragment:

An anti-E tag antibody immunoaffinity column was prepared by amine coupling to protein-G sepharose using dimethyl-pimilimidate (DMP). Briefly, 1.5 mg of anti-E-tag antibody was covalertly coupled to 1.0 ml of protein-G sepharose using 20 mM DMP. After blocking and regeneration of the matrix, it was used for binding and purification of ScFv:GyrA. The 30-70% ammonium sulfate fraction from culture supernatant containing soluble scFv was dialyzed against PBS and loaded onto the anti-E tag column. After washing the column with Glycine-HCl pH 5.0 buffer, scFv was eluted with Glycine-HCl pH 2.8. The eluted fractions were immediately neutralized with Tris-HCl pH 9.0. The fractions were further analyzed on 12% SDS-PAGE and followed by silver staining. The fractions containing scFv were pooled and dialyzed against Tris-KCl buffer (Tris-HCl 35 mM, 50 mM KCl and 10% Glycerol).

E7: Kinetics of scFv:GyrA Interaction with M. smegmatis GyrA and Its Comparison with IgG and Fab Fragments:

Binding parameters were determined by Surface Plasmon Resonance (SPR) spectroscopy using a BIAcore 2000 system, Uppsala, Sweden. The over-expressed and purified M. smegmatis GyrA was immobilized on the CM5 sensor chip at a concentration of 400 resonance units (RU) as per manufacturer's instructions. All measurements were carried out in a continuous flow of HBS buffer (10 mM HEPES, 150 mM NaCl, 5 mM EDTA and 0.05 % Surfactant P-20, pH 7.4) at 10 μl/min. Purified MsGyrA:C3 IgG and its Fab and scFv:GyrA fragments were dialyzed against HBS buffer and used for interaction studies. The kinetic data were analyzed using the BIA evaluation software (Version 3.0). The surface was regenerated by a pulse of 5 μL of 100 mM NaOH for further experimentation as required.

E8: DNA and Protein Sequencing Analysis:

Sequencing of the two independent scFv clones was carried out using 5′ and 3′ pCANTAB sequencing primers. The nucleotide sequence of two of the scFv:GyrA clones were translated to amino acid sequence and analyzed using DNA analysis program. The immunoglobulin sequence was further analyzed using Kabat database (E. A. Kabat et. al., Sequences of proteins of immunologic interest, United states Department of Health and Human services, Public health service, National institute of Health publication No. 37, 1987) to define frame work and hypervariable regions. For N-terminal amino acid sequencing of MsGyrA:C3 K chain, proteins were transferred onto an immobilon PVDF membrane (Millipore). The n-terminal amino acid sequence was determined by automated Edman degradation methodology and found to be IVMTQSPKS, confirming the authenticity of scFv sequence.

E9: Experiment to Show Affinity of Interaction and Inhibition of DNA Supercoiling Activity bay IgG, Fab and scFv are Similar

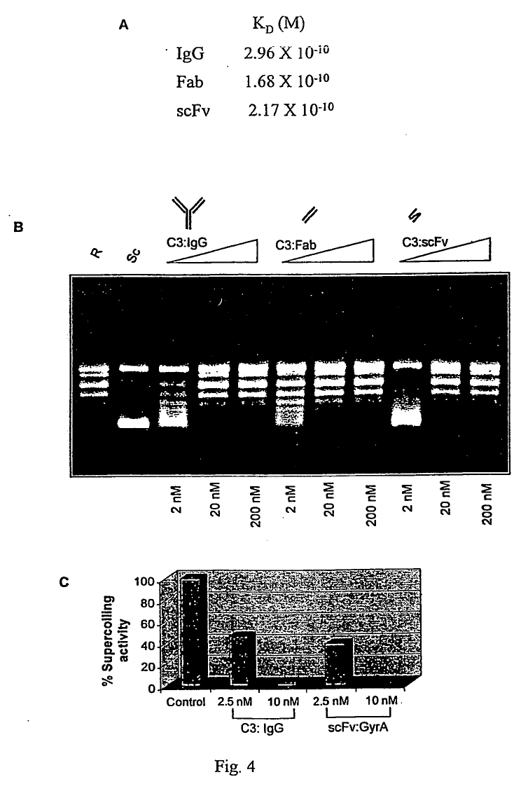

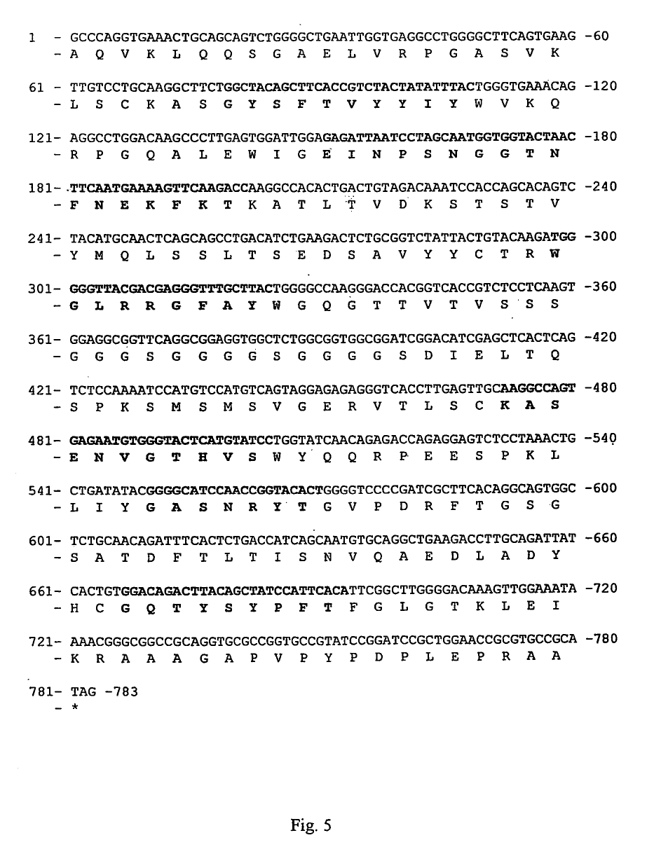

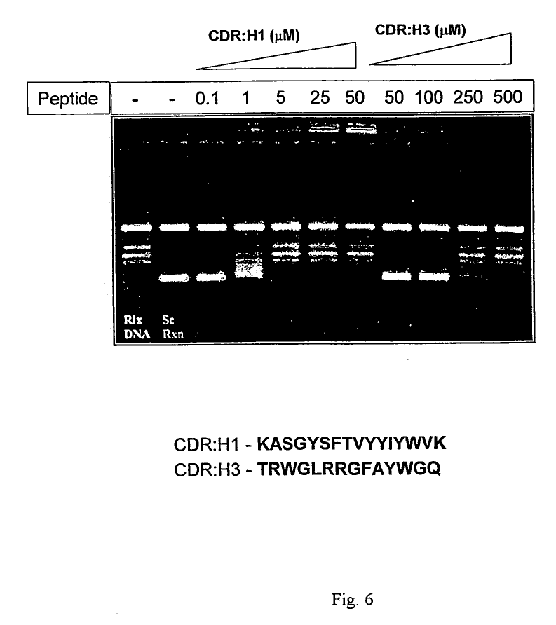

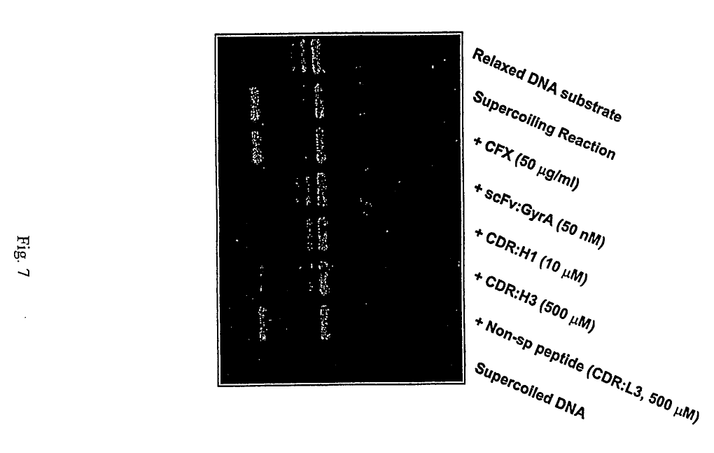

In order to measure the affinity of interaction of scFv:GyrA and to compare it with C3:IgG and C3:Fab, surface plasmon resonance studies were carried out on GyrA immobilized surface. The scFv:GyrA interacted with M. smegmatis GyrA with an affinity of 2.17×10⁻¹⁰ M (FIG. 4A), which is similar to its parent bivalent IgG (2.96×10⁻¹⁰ M) or monovalent Fab (1.68×10⁻¹⁰ M). This emphasizes that the engineering of two variable domains into a single polypeptide did not alter the affinity of interaction of antibody with its antigen.

To study the effect of scFv:GyrA interaction with GyrA on enzymatic activity, DNA supercoiling reactions were performed with M. smegmatis enzyme in presence of scFv:GyrA or parental IgG or Fab fragments. The enzyme was pre-incubated with soluble scFv, IgG or Fab to allow the formation of the antigen-antibody complex The mixture was then added to relaxed pUC18 DNA, in the supercoiling reaction buffer. The results (FIG. 4B) showed that mAb:C3 IgG, Fab and scFv fragments inhibited the mycobacterial DNA gyrase supercoiling activity to a similar extent. As compared to IgG, which is a 150 kDa protein with two antigen binding pockets, scFv is a monovalent 30 kDa polypeptide. The scFv fragments inhibited the supercoiling reaction at a concentration of 10-20 nM, which is in the same range as that of IgG or Fab. This indicates that inhibition is caused by direct interaction of the antigen binding site of the antibody with GyrA, rather than a result of a steric effect caused by the Fc regions of the inmmunoglobulins or by cross linking of adjacent GyrA molecules. DNA gyrase from ofloxacin resistant, highly virulent clinical isolate of M. tuberculosis (ICC-222) was also completely inhibited by 10 nM scFv:GyrA (FIG. 4C) like its parent IgG.

E10: Experiment to Show the scFv:GyrA Sequence and Identification of Complementarity Determining Regions

To characterize the structural features of MsGyrA:C3 responsible for their interactions with GyrA subunit, both heavy and light chain variable regions were sequenced (Seq. ID #1 and 2). Comparison of the sequence with other known antibody sequences showed that V_{H} region of MsGyrA:C3 belongs to subgroup IIIB according to the classification of E. A. Kabat et. al., Sequences of proteins of immunologic interest, United states Department of Health and Human services, Public health service, National institute of Health publication No. 37, 1987. Sequence analysis of V_{L} region suggest that it belongs to subgroup V. Based on the sequence analysis as defined by Kabat database the complementarity determining regions for light and heavy chain regions of scFv:GyrA were identified and shown in bold letters in Seq. IDs #1 and 2.

E11: Experiment to Show Inhibition by Paratope Derived Peptides

An antibody molecule binds specifically to its cognate antigen by synergistically using multiple non-covalent forces. The diversity of paratopes is mainly generated by sequences present in the complementarity determining regions (CDR) of V_{H} (Variable heavy chain) and V_{L}, (Variable light chain) which are exposed hypervariable loop structures, Antigen binding by peptide sequences from selected CDRs of mAbs have been demonstrated to have specificity similar to those of the original antibody molecule. This section deals with rational design of biologically active peptides from the sequence of scFv:GyrA with mycobacterial DNA gyrase inhibitory activity.

Based on variable sequence of scFv:GyrA, peptides corresponding to CDRs were synthesized and analyzed for DNA gyrase inhibition. We have identified a CDR:H1 sequence that specifically inhibits the DNA gyrase supercoiling activity. The results clearly indicate that the sequence KASGYSFTVYYIYWVK in CDR-H1 is a major part of the discontinuous determinant involved in the antigen-antibody interaction. The complete inhibition of enzyme activity was observed at 5-10 μM peptide concentration in contrast to >500 μM of CDR:H3 (Seq. IDs #1 and 2 ). The results also suggest that other such inhibitory peptides could be designed using other CDR and framework region sequence.

E12: Experiment to Show scFv:GyrA and the Peptides Derived from scFv Inhibit Ciprofloxacin Resistant Mycobacterial DNA Gyrase

The experiments presented in this section of the invention address the inhibition of quinolone resistant DNA gyrase activity by scFv:GyrA, CDR:H1 and CDR:H3 peptides. DNA gyrase from ciprofloxacin resistant (MIC₅₀ 64 μg/ml) M. smegmatis mc²155 strain was used for the experiments. As expected, the enzyme showed resistance to ciprofloxacin (50 μg/ml) (Seq. ID #1 and 2). However, DNA supercoiling activity was inhibited at 50 nM of scFv:GyrA and 10 μM CDR:H1 peptide (Seq. ID #1 and 2). Thus, the peptide inhibitor is equally effective against quinolone resistant DNA gyrase.

1. An engineered single chain antibody, which inhibits the activity of DNA gyrase from M. smegmatis and M. tuberculosis.

2. The engineered single chain antibody as claimed in claim 1 wherein it contains amino acid sequences for inhibiting the activity of DNA gyrase from M. smegmatis and M. tuberculosis said amino acid sequences having the Seq. ID #3 and 4 respectively.

3. An engineered single chain antibody as claimed in claim 1 wherein said antibody has a nucleotide sequence shown in Seq. ID #1.

4. An engineered single chain antibody as claimed in claim 1 wherein said antibody has an amino acid sequence shown in Seq. ID #2.

5. A peptide having an amino acid sequence as shown in Seq. ID #2.

6. A process for the preparation of an engineered single chain antibody which inhibits the activity of DNA gyrase from M. smegmatis and M. tuberculosis, said process comprising preparing complimentary DNA (cDNA) from the corresponding hybridoma cell lines which secretes monoclonal antibody, amplifying from said cDNA, DNA fragments encoding variable heavy chain region and light regions of said monoclonal antibody, fusing said variable heavy chain and light chain regions of said DNA fragments, cloning said fused DNA fragment in a plasmid, transforming said plasmid into E. Coli host strain, inducing said transformed cells to express said engineered single chain antibody and purifying said engineered single chain antibody from the induced cell lysate.

7. Monoclonal antibodies, which inhibit DNA gyrase from fluoroquinolone resistant M. smegmatis and M. tuberculosis.

8. A plasmid characterised in that it encodes an engineered single chain antibody containing amino acid sequences for inhibiting the activity of DNA gyrase from M. smegmatis and M. tuberculosis, said amino acid sequences being as shown in Seq. ID #3 and 4 respectively.
